(19) United States
(12) Patent Application Publication
(10) Pub. No.: US 2017/0192000 A1
(43) Pub. Date: Jul. 6, 2017

(54) METHOD FOR MONITORING CATHEPSIN S INHIBITION

(71) Applicant: Hoffmann-La Roche Inc., Little Falls, NJ (US)

(72) Inventors: Ana Patricia BENTO PEREIRA DA SILVA, Basel (CH); Sebastian DZIADEK, Benediktbeuern (DE); Barbara ECABERT, Therwil (CH); Michael GERG, Muenchen (DE); Everson NOGOCEKE, Basel (CH); Moritz MARCINOWSKI, Mannheim (DE); Bernhard REIS, Basel (CH); Thomas SCHINDLER, Loerrach (DE); Michel Achille Edouard THERON, Ruelisheim (FR)

(73) Assignee: Hoffmann-La Roche Inc., Little Falls, NJ (US)

(21) Appl. No.: 15/456,353

(22) Filed: Mar. 10, 2017

Related U.S. Application Data
(63) Continuation of application No. PCT/EP2015/071002, filed on Sep. 15, 2015.

(30) Foreign Application Priority Data
Sep. 18, 2014 (EP) 14185409.1

(51) Int. Cl. G01N 33/569; C07K 16/28

(52) U.S. Cl. CPC G01N 33/56972; C07K 16/2833; G01N 2333/70539; G01N 2333/8139; C07K 2317/565; C07K 2317/56; G01N 2500/10; G01N 2500/02

(57) ABSTRACT

The present invention relates to a method for monitoring Cathepsin S inhibitor activity in tissue samples of animals comprising:
a) providing a tissue sample of an animal to whom a Cathepsin S inhibitor has been administered or providing a tissue sample of an animal that was contacted in vitro with a Cathepsin S inhibitor, wherein the tissue samples comprise white blood cells,
b) measuring li p10 peptide level in white blood cells of the tissue sample of step a) by flow cytometry and
c) correlating li p10 peptide level in white blood cells to Cathepsin S inhibitor dose, wherein a Cathepsin S inhibitor leads to increased level of li p10 peptide in the white blood cells.

### CROSS REFERENCE TO RELATED APPLICATIONS

This application claims priority to PCT Application No. PCT/EP2015/071002, filed on Sep. 15, 2015, which claims priority to European patent application no. 14185409.1, filed on Sep. 18, 2004, the entire contents of which is incorporated herein by reference.

### SUBMISSION OF SEQUENCE LISTING ON ASCII TEXT FILE

The instant application contains a Sequence Listing which has been submitted electronically in ASCII format and is hereby incorporated by reference in its entirety. Said ASCII copy, created on Feb. 23, 2017 is named P32272-US_SeqListing.txt and is 9,793 bytes in size.

### BACKGROUND OF THE INVENTION

The present invention provides an antibody directed to li p10 peptide and its use in methods for monitoring Cathepsin S inhibition.

Cathepsin S is well known for its critical function in the proteolytic digestion of the invariant chain chaperone molecules, thus controlling antigen presentation to CD4⁺ T-cells by major histocompatibility complex (MHC) class II molecules or to NK1.1+ T-cells via CD1 molecules. Cathepsin S also appears to participate in direct processing of exogenous antigens for presentation by MHC class II to CD4⁺ T-cells, or in cross-presentation by MHC class I molecules to CD8⁺ T-cells. In addition, in its secreted form Cathepsin S is implicated in degradation of the extracellular matrix, which may contribute to the pathology of a number of diseases, including arthritis, atherosclerosis and chronic obstructive pulmonary disease. Therefore, inhibition of Cathepsin S is a promising target for the development of novel therapeutics for a variety of indications.

MHC class II molecules bind and display antigenic peptides as class II-peptide complexes on the cell surface of antigen presenting cells (APCs) for recognition by CD4⁺ helper cells. The molecular mechanisms leading to formation of class II-peptide complexes and the presentation of antigens on the cell surface begin with synthesis of class II αβ heterodimers in the endoplasmatic reticulum. During biosynthesis these αβ heterodimers associate with a type II membrane protein, the isoforms of the invariant chain li, to form a nonameric complex of αβli. The αβli complex traverses the Golgi apparatus and is delivered to intracellular compartments. In these acidic compartments, the li is degraded and finally liberated from the αβli complexes, allowing class II molecules to bind antigenic peptides. The proteolytical processing of li results in shorter fragments. The cysteine protease Cathepsin S plays a key role in processing li in APCs. Cathepsin S degrades the invariant chain li from class II-li complexes generating class II-CLIP. Hence, inhibition of Cathepsin S leads to a decrease of CLIP peptide and to accumulation of the precursor fragment, i.e. the invariant chain li degradation peptide p10 (li p10).

EP 1 315 966 discloses a method for monitoring the effect of in vivo administration of cathepsin S inhibitors. The method includes the purification of white blood cells from a blood sample of a subject treated with a Cathepsin S inhibitor, preparation of whole cell lysates of the purified white blood cells and analyzing the whole cell lysates for presence of li p10 fragment of invariant chain li. This method requires blood samples of about 10-30 ml and is labour intensive (isolation of white blood cells and preparation of whole cell lysates). The detection of li p10 is done by ELISA or Western blot.

The aim of the present invention is to provide an assay to monitor Cathepsin S inhibition which overcomes at least some of the drawbacks of the prior art methods.

In a first object, the present invention provides a method for monitoring Cathepsin S inhibitor activity in tissue samples of animals comprising:

a) providing a tissue sample of an animal to whom a Cathepsin S inhibitor has been administered or providing a tissue sample of an animal that was contacted in vitro with a Cathepsin S inhibitor, wherein the tissue sample comprises white blood cells,

b) measuring lip10 peptide level in white blood cells of the tissue sample of step a) by flow cytometry and

c) correlating li p10 peptide level in white blood cells to Cathepsin S inhibitor dose, wherein a Cathepsin S inhibitor leads to increased level of li p10 peptide in white blood cells.

In a second object the present invention provides a method for the identification of a Cathepsin S inhibitor comprising:

a) treating a non-human animal with a test compound,

b) providing a tissue sample of the animal of step a) comprising white blood cells,

c) measuring lip10 peptide level in the white blood cells of the tissue sample of step b),

wherein an increased level of li p10 peptide in white blood cells in the sample of step a) compared to a level of li p10 peptide in white blood cells in a tissue sample of an untreated animal is indicative for a Cathepsin S inhibitor.

In a preferred embodiment of the methods of the present invention the tissue sample is blood.

In a further preferred embodiment of the methods of the present invention the white blood cells are Antigen Presenting Cells (APCs), preferably the APCs are selected from B cells, monocytes, macrophages and dendritic cells, more preferably selected from B cells and monocytes.

The term Antigen Presenting cells (APCs) as used herein refers to cells that display antigenic peptides as (MHC) class II-antigenic peptide complex on its surface. APCs comprise dendritic cells, monocytes, macrophages and B cells.

In a third object, the present invention provides an anti-li p10 antibody comprising:

a VH domain comprising a CDR1 comprising the amino acid sequence of Seq. Id. No. 4, a CDR2 comprising the amino acid sequence of Seq. Id. No. 5, CDR3 sequence comprising the amino acid sequence of Seq. Id. No. 6., and a VL domain comprising a CDR1 comprising the amino acid sequence of Seq. Id. No. 7, a CDR2 comprising the amino acid sequence of Seq. Id. No. 8, a CDR3 sequence comprising the amino acid sequence of Seq. Id. No. 9.

In a particular embodiment of the present invention, the VH domain of the anti-li p10 antibody comprises the amino acid sequence of Seq. Id. No. 10 and the VL domain of the anti-li p10 antibody comprises the amino acid sequence of Seq. Id. No. 11.

The inventive anti-li p10 specific antibody is referred to herein as 7B8 neoepitope antibody originating from Oryctologous cuniculus. This antibody is characterized by CDRs and VH, VL sequences disclosed in Seq. Id. Nos. 4-11.

The term Cathepsin S refers to a cysteine protease in the papain super-family. The amino acid sequence of human Cathepsin S is given in Seq. Id. No. 1.

The term “invariant chain li” refers to invariant polypeptide of major histocompatibility complex or CD74. The amino acid sequence of human invariant chain li is given in Seq. Id. No. 2.

The term li p10 peptide refers to class II-associated li peptide. The amino acid sequence of human li p10 is given in Seq. Id. No. 3.

The term “MHC II polypeptide” as used herein refers to the alpha chain and the beta chain constituting the MHC II complex.

The term animal as used herein comprises human beings and non-human animals such as e.g. monkey, dog, pig, rabbit, guinea pig and rodents.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The inventors of the present invention have surprisingly found that li p10 peptide can be detected in white blood cells. The methods of the present invention have the advantage compared to the methods described in the prior art that tissue samples, preferably whole blood, can be assayed directly without involving the isolation of white blood cells. In the methods of the present invention there is no need for the isolation of e.g. white blood cells from whole blood samples, in order to be able to be processed in a method of the present invention. This is of great importance if the assay is used in clinical settings where a large number of samples have to be processed. The methods of the present invention require smaller tissue samples, preferably blood samples, than the prior art methods.

Methods for detection and/or measurement of polypeptides in biological samples are well known in the art and include, but are not limited to, Western-blotting, Flow cytometry, ELISAs or RIAs, or various proteomics techniques. Monoclonal or polyclonal antibodies recognizing the li p10 peptide or fragments thereof, can either be generated for the purpose of detecting the polypeptides or peptide fragments, e.g. by immunizing rabbits with purified proteins, or known antibodies recognizing the polypeptides or peptide fragments can be used. For example, an antibody capable of binding to the denatured proteins, such as a polyclonal antibody, can be used to detect LI P10 peptide or MHC II polypeptide in a Western Blot. An example for a method to measure a polypeptide is an ELISA. This type of protein quantitation is based on an antibody capable of capturing a specific antigen, and a second antibody capable of detecting the captured antigen. Methods for preparation and use of antibodies, and the assays mentioned hereinbefore are described in Harlow, E. and Lane, D. Antibodies: A Laboratory Manual, (1988), Cold Spring Harbor Laboratory Press.

A preferred method for the detection of li p10 peptide is flow cytometry. Flow cytometry methods are described in Handbook of Flow Cytometry Method, J. Paul Robinson (Editor); Flow Cytometry—A Basic Introduction, Michael G Ormerod (2008) and Current Protocols in Cytometry (2010), Wiley. In this method proteins are not denatured but preserved in their native form.

WO 2010/121918 discloses Cathepsin S inhibitors which can be tested in the method of the present invention.

### SHORT DESCRIPTION OF THE FIGURES

FIG. 1: Detection of Ii p10 expression on B cells and T cells using a neoepitope antibody. PBMCs isolated from blood donation of a healthy donor (R213) were incubated for 20 h with increasing concentrations of Cathepsin S inhibitor 1 or vehicle. Ii p10 detection on B cells (CD20+) and T cells (CD3+) was determined by flow cytometry using a different neoepitope antibody 7B8 (black).

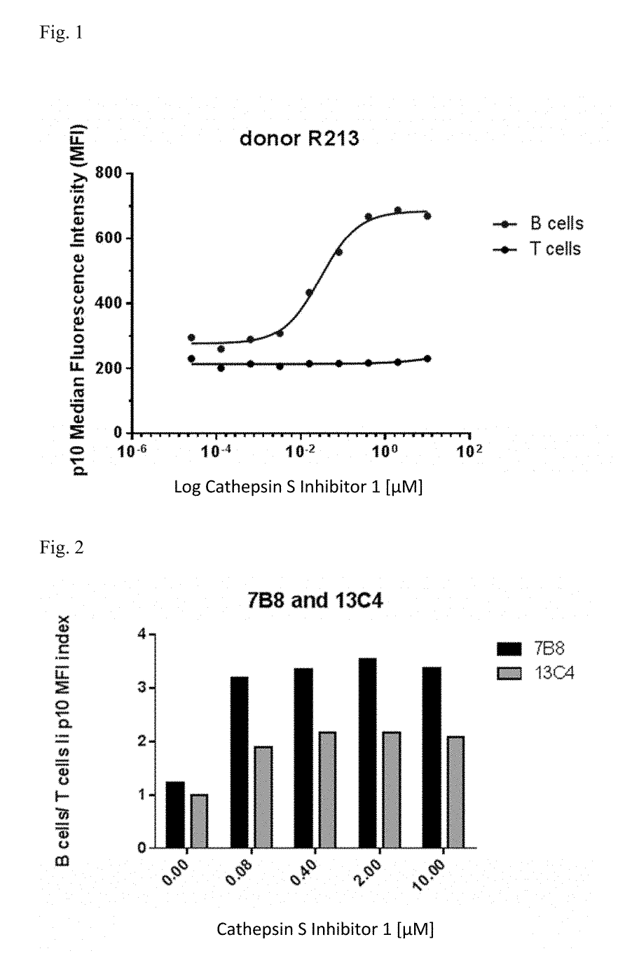

FIG. 2: Detection of Ii p10 expression on B cells using a neoepitope antibody. PBMCs isolated from blood donation of a healthy donor (R172) were incubated for 20 h with increasing concentrations of Cathepsin S inhibitor 1 or vehicle. Ii p10 detection on B cells (CD20+) and T cells (CD3+) was determined by flow cytometry using two different neoepitope antibodies, namely 7B8 (black) and 13C4 (grey). Stain index=Median Fluorescence Intensity (MFI)_{B cells}/MFI_{Tcells}

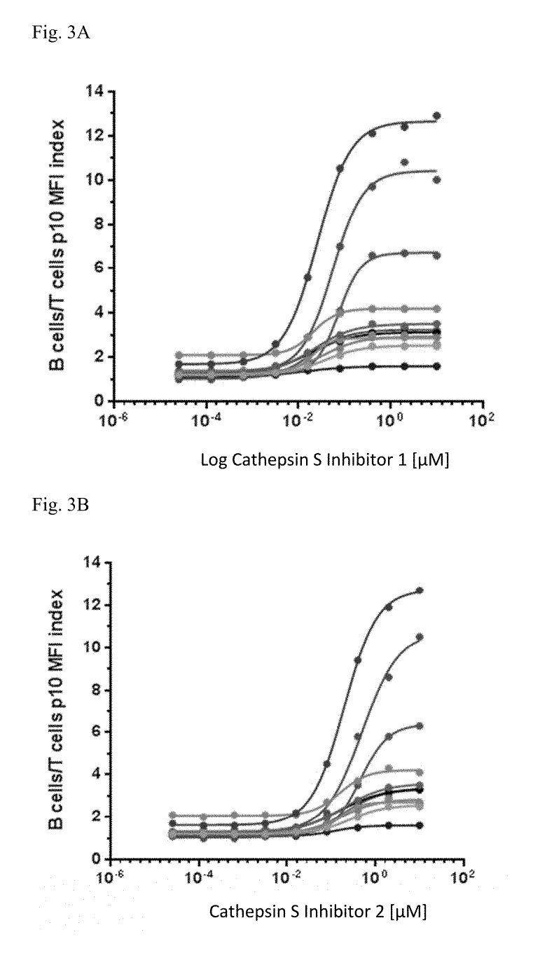

FIG. 3 A: Ii p10 accumulates in B cells upon treatment with Cathepsin S inhibitors. PBMCs isolated from blood donations of eleven healthy donors (R39; R185; R198; R204; R209; R140; R154; R175; R213; R4;R214) were incubated for 20 h with increasing concentrations of Cathepsin S inhibitor 1 or vehicle. Ii p10 detection on B cells (CD20+) and T cells (CD3+) was determined by flow cytometry using 7B8 neoepitope antibody. Stain index=MFI_{B cells}/MFI_{Tcells}.

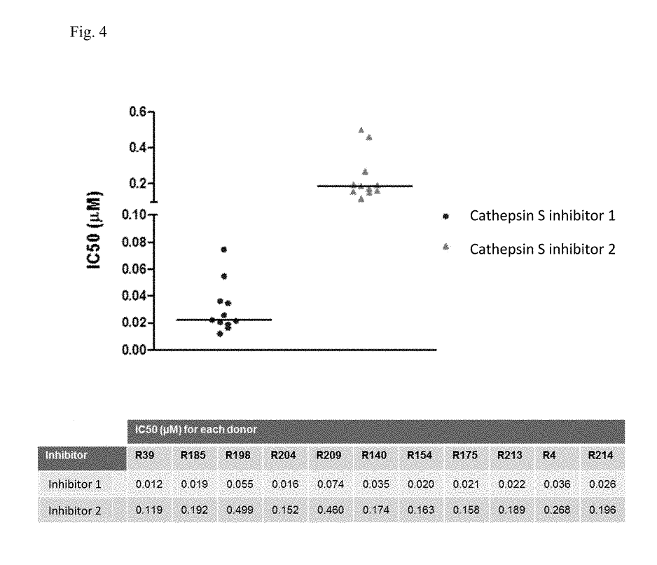

FIG. 3 B: Ii p10 accumulates in B cells upon treatment with Cathepsin S inhibitors. PBMCs isolated from blood donations of eleven healthy donors (R39; R185; R198; R204; R209; R140; R154; R175; R213; R4;R214) were incubated for 20 h with increasing concentrations of Cathepsin S inhibitor 2 or vehicle. Ii p10 detection on B cells (CD20+) and T cells (CD3+) was determined by flow cytometry using 7B8 neoepitope antibody. Stain index=MH_{B cells}/MFI_{Tcells}.

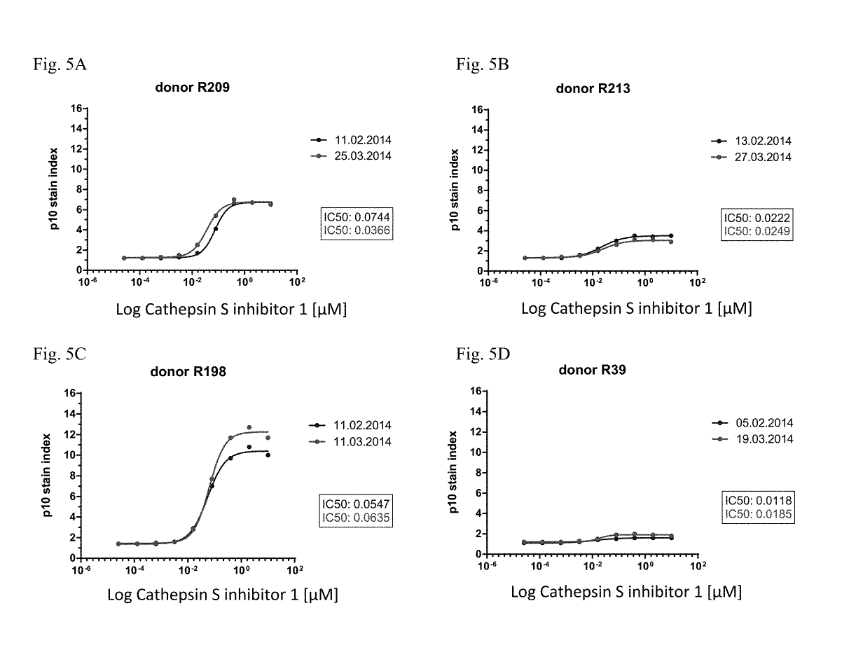

FIG. 4: Cathepsin S inhibitor IC50 concentrations. PBMCs isolated from blood donations of eleven healthy donors (R39; R185; R198; R204; R209; R140; R154; R175; R213; R4;R214) were incubated for 20 h with increasing concentrations of Cathepsin S inhibitor 1, Cathepsin S inhibitor 2 or vehicle. Ii p10 detection on B cells (CD20+) and T cells (CD3+) was determined by flow cytometry using 7B8 neoepitope antibody. Response curves and IC50 values have been calculated with GraphPad Prism software.

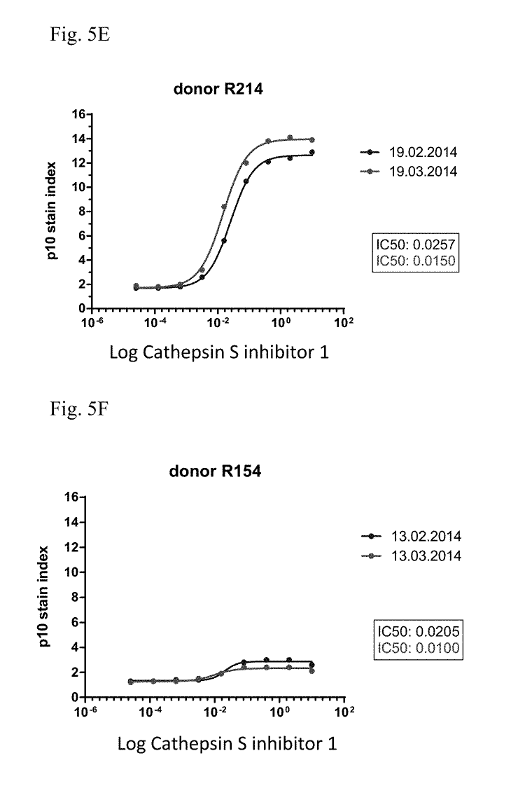

FIGS. 5 A-F: Ii p10 neoepitope assay longitudinal variability. PBMCs isolated from blood donations of eleven healthy donors (R198; R209; R154; R213; R214) at two time points and were incubated for 20 h with increasing concentrations of Cathepsin S inhibitor 1, or vehicle. Ii p10 detection on B cells (CD20+) and T cells (CD3+) was determined by flow cytometry using 7B8 neoepitope antibody.

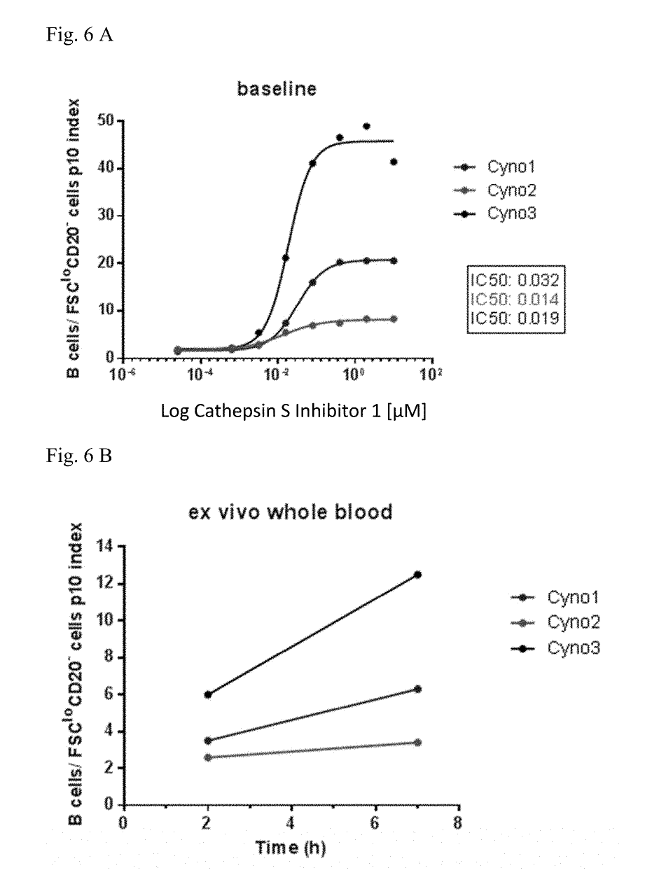

FIG. 6 A: Ii p10 accumulation in B cells is detected by the neoepitope assay. Ii p10 detection on B cells (CD20+) was determined by flow cytometry using 7B8 neoepitope antibody clone. PBMCs isolated from 3 cynos (Macaca fascicularis) blood donations were incubated for 20 h with increasing concentrations of Cathepsin S inhibitor 1 or vehicle. The p10 stain index of Macaca fascicularis samples was determined by dividing the Median Fluorescence Intensity (MFI) of B cells by the MFI of Forward Scatter Low CD20 negative (FSCloCD20−) cells.

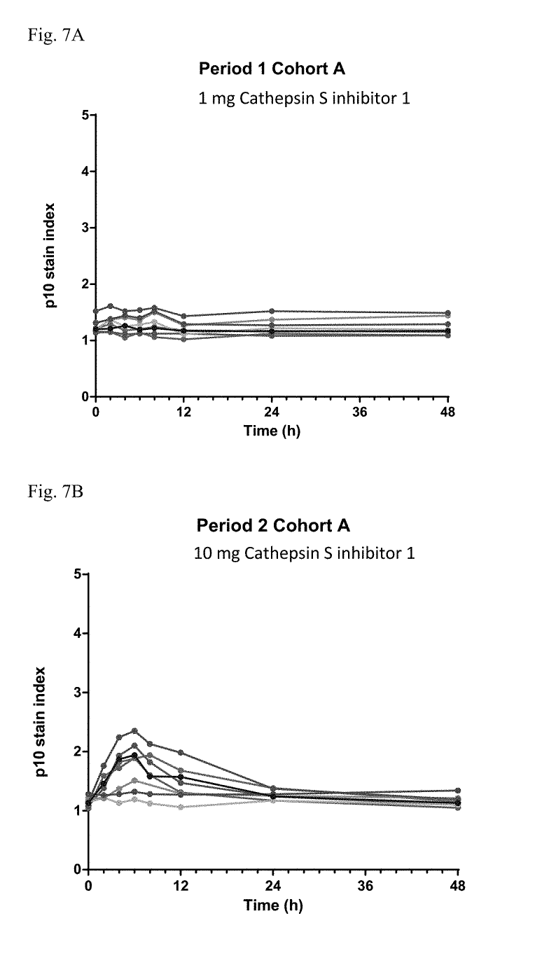

FIG. 6 B: Ii p10 accumulation in B cells is detected by the neoepitope assay after a single oral dose of Cathepsin S inhibitor. Ii p10 detection on B cells (CD20+) was determined by flow cytometry using 7B8 neoepitope antibody clone. Blood was collected from animals 2 h and 7 h after a single dose of 50 mg/kg. Ii p10 neoepitope assay was performed in whole blood. The p10 stain index of Macaca fascicularis samples was determined by dividing the Median Fluorescence Intensity (MFI) of B cells by the MFI of Forward Scatter Low CD20 negative (FSCloCD20−) cells.

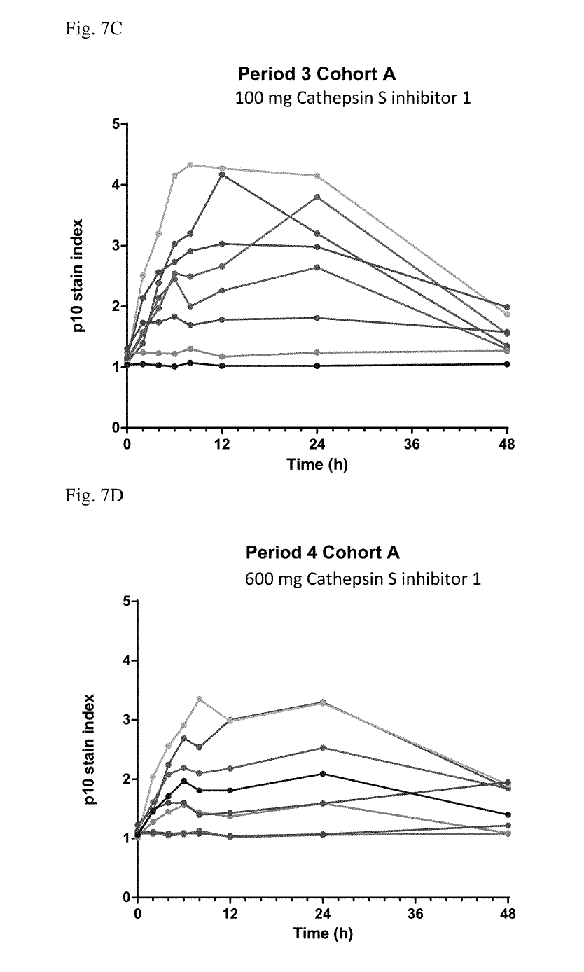

FIG. 7A-7D: Quantification of p10 on samples obtained from subjects participating in a single center, randomized, double-blind, placebo-controlled, single ascending dose study to evaluate the safety, tolerability, pharmacokinetic and pharmacodynamic effects of single doses of Cathepsin inhibitor 1 in healthy male and female volunteers. To account for and limit the impact of the expected inter-subject variability as observed in preclinical experiments, an interleaved cohort (“leapfrog”) design was employed. Subjects were recruited into two cohorts of eight subjects each (Cohorts A and B). For each individual within a cohort, the study was a randomized, placebo controlled, four treatment, four periods, four-way crossover. The single dose applied shortly after time point 0 hours is indicated on each FIG. 7A to 7D (cohort A) and FIG. 8A to 8D (cohort B).

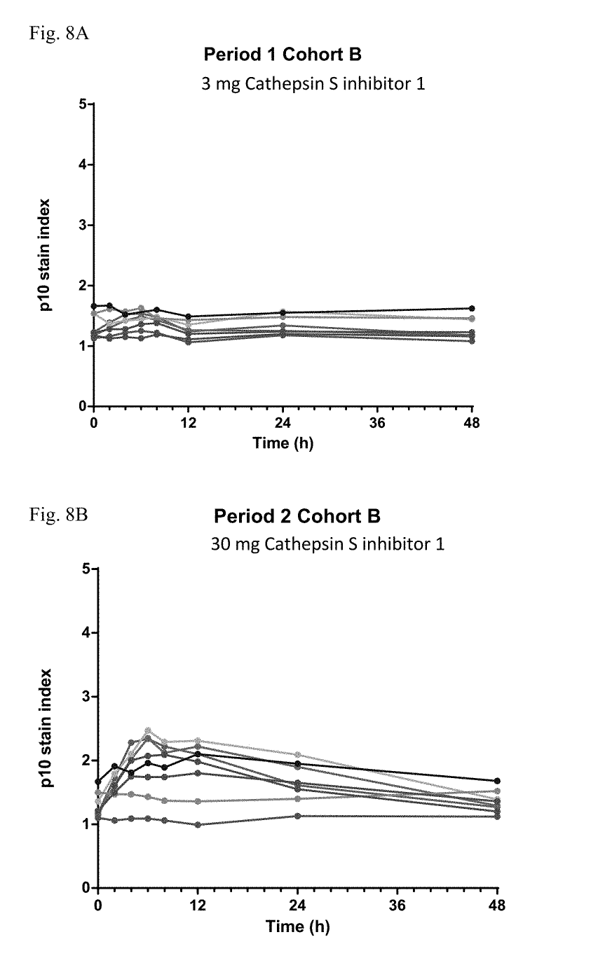

FIG. 8A-8D: Cohort B data, see FIG. 7 description.

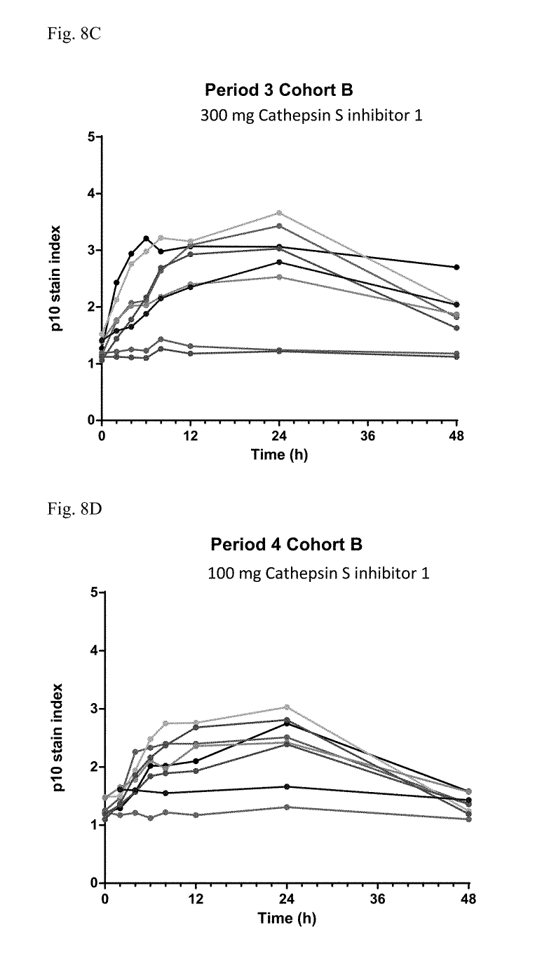

### EXPERIMENTAL PART

In Vitro Assay with Cathepsin S Inhibitor
Use a sterile Polystyrene 24 well plate
Add 500 μl complete RPMI+Cathepsin S inhibitor with increasing concentrations (0 to 10 μM)
Add 500 μl of PBMC suspension (2×10⁶) to each well
Mix the content of the wells and incubate the plate for 20 h at +37° C., 5% CO2 in a humidified incubator

### FACS Staining:

prepare 1× lyse/fix solution
add 4 mL of pre-warmed 1× lyse/fix solution per 2×10⁶ PBMCs or 200 μl blood
incubate for 10 min at 37 C, wash the cells 1× with PBS (250×g, 5 minutes, RT), remove supernatant
add 2 mL of 1× permeabilising solution per tube
incubate for 20 min at RT
wash the cells 2× with 2 ml Cell Stain Buffer (250×g, 5 minutes, RT), remove supernatant
add 10 μg/mL unlabeled anti-p10 antibody in Cell Staining Buffer for 1 h at RT
wash the cells 1× with 2 ml Cell Stain Buffer (250×g, 5 minutes, RT), remove supernatant
add 1:125 PE-conjugated goat anti-rabbit IgG antibody in Cell Staining for 30 min at RT in the dark
wash the cells 2× with 2 ml Cell Stain Buffer (250×g, 5 minutes, RT), remove supernatant
add the antibodies (comp. control) or antibody mixture (diluted in 100 μl/sample Cell Stain Buffer) to each tube
incubate for 20 min at +4° C. in the dark
wash the cells 1× with 2 ml Cell Stain Buffer (250×g, 5 minutes, RT), remove supernatant
resuspend pellet in 200 μl Cell Stain Buffer and keep the cells in the dark until the analysis on the FACS

Antibodies:

| | | |
|---|---|---|
| goat anti-rabbit IgG PE | SouthernBiotech | Cat# 4050-09 |
| | | 1:125/sample |
| CD3 Brilliant Violet 421 | BioLegend | Cat# 300434 |
| | | 5 μl/sample |
| CD14 APC | Beckman Coulter | Cat# IM2580 |
| | | 5 μl/sample |
| CD20 AlexaFluor 488 | BD Biosciences | Cat# 558056 |
| | | 20 μl/sample |
| CD45 PerCP/Cy5.5 | BioLegend | Cat# 304028 |
| | | 2 μl/sample |

Materials and Buffers:

24-well plates, BD, order ID: 351147Storage: room temperature, sterile, polystyrene non tissue culture treated

10× BD Phosphoflow Lyse/Fix buffer lysis buffer (BD, order ID: 558049). Storage: +4° C. Dilute the 5× BD Phosphoflow Lyse/Fix buffer with H₂O bidest. to 1× and prewarm it to the required temperature prior to use 10× Phosflow Perm Buffer IV permeabilisation buffer (BD, order ID: 560746). Storage at RT. Dilute 10× BD Phosphoflow Perm Buffer IV with PBS to 1×.

1× Cell Satining Buffer (BioLegend, order ID: 420201. Storage: +4 C the 1× Cell Satining Buffer is ready to use. 1× PBS without CaCl₂ and without MgCl₂ (Gibco, order ID: 14190-094). Storage: room temperature. The PBS is ready to use. 5 ml Polystyrene round-bottom tube (FACS tube, BD, order ID: 352052). Storage: room temperature.

### Amino Acid Sequences

| Sequence name | Seq. Id. No. |
|---|---|
| Human Cathepsin S | 1 |
| Human invariant chain li | 2 |
| Human li p10 | 3 |
| VH CDR 1 of anti li p10 antibody 7B8 | 4 |
| VH CDR 2 of anti li p10 antibody 7B8 | 5 |
| VH CDR 3 of anti li p10 antibody 7B8 | 6 |
| VL CDR 1 of anti li p10 antibody 7B8 | 7 |
| VL CDR 2 of anti li p10 antibody 7B8 | 8 |
| VL CDR 3 of anti li p10 antibody 7B8 | 9 |
| VH chain of anti li p10 antibody 7B8 | 10 |
| VL chain of anti li p10 antibody 7B8 | 11 |

1. A method for monitoring Cathepsin S inhibitor activity in a tissue sample of animals comprising:
a) providing a tissue sample of an animal to whom a Cathepsin S inhibitor has been administered or providing a tissue sample of an animal that was contacted in vitro with a Cathepsin S inhibitor, wherein the tissue sample comprise white blood cells;
b) measuring lip10 peptide level in white blood cells of the tissue sample of step a) by flow cytometry; and
c) correlating li p10 peptide level in white blood cells to Cathepsin S inhibitor dose, wherein a Cathepsin S inhibitor leads to increased level of li p10 peptide in white blood cells.

2. A method for the identification of a Cathepsin S inhibitor comprising:
a) contacting a non-human animal with a test compound;
b) providing a tissue sample of the animal of step a) comprising white blood cells; and
c) measuring li p10 peptide level in white blood cells of the tissue sample of step b) by flow cytometry;
wherein an increased level of li p10 peptide in white blood cells in the sample of step a) compared to a level of li p10 peptide in white blood cells in a tissue sample of an untreated animal is indicative for a Cathepsin S inhibitor.

3. The method of claim 1 wherein the tissue sample is blood.

4. The method of claim 2 wherein the tissue sample is blood.

5. The method of claim 1, wherein the white blood cells are Antigen Presenting Cells (APCs).

6. The method of claim 2, wherein the white blood cells are Antigen Presenting Cells (APCs).

7. The method of claim 5, wherein the APCs are selected from B cells and monocytes.

8. The method of claim 6, wherein the APCs are selected from B cells and monocytes.

9. The method of claim 1 wherein the animal is a human being.

10. The method of claim 2, wherein the non-human animal is a rodent.

11. The method of claim 1, wherein the white blood cells are permeabilized to measure li p10 peptide by flow cytometry.

12. The method of claim 2, wherein the white blood cells are permeabilized to measure li p10 peptide by flow cytometry.

13. The method of claim 1, wherein an antibody directed to li p10 is used in flow cytometry to measure li p10 in white blood cells.

14. The method of claim 2, wherein an antibody directed to li p10 is used in flow cytometry to measure li p10 in white blood cells.

15. An anti li p10 antibody comprising a VH domain comprising a CDR1 comprising the amino acid sequence of SEQ ID NO:4, a CDR2 comprising the amino acid sequence of SEQ ID NO:5, CDR3 sequence comprising the amino acid sequence of SEQ ID NO:6., and a VL domain comprising a CDR1 comprising the amino acid sequence of SEQ ID NO:7, a CDR2 comprising the amino acid sequence of SEQ ID NO:8, a CDR3 sequence comprising the amino acid sequence of SEQ ID NO:9.

16. The anti li p10 antibody of claim 9, wherein the VH domain comprises the amino acid sequence of SEQ ID NO:10 and the VL domain comprises the amino acid sequence of SEQ ID NO:11.

17. The method of claim 13, wherein said antibody is the antibody of claim 15.

18. The method of claim 13, wherein said antibody is the antibody of claim 16.

19. The method of claim 14, wherein said antibody is the antibody of claim 15.

20. The method of claim 14, wherein said antibody is the antibody of claim 16.


SEQUENCE LISTING (11 sequences)

SEQ ID NO: 1 - protein, 331 aa, Homo sapiens [sequence omitted]

SEQ ID NO: 2 - protein, 296 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 3 - protein, 104 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPV

SEQ ID NO: 4 - protein, 10 aa, Oryctolagus cuniculus
GIDLSTYAMT

SEQ ID NO: 5 - protein, 14 aa, Oryctolagus cuniculus
IIYNNGVVVYASWA

SEQ ID NO: 6 - protein, 9 aa, Oryctolagus cuniculus
GYYDGSVNV

SEQ ID NO: 7 - protein, 13 aa, Oryctolagus cuniculus
QSSQSVYNDNVLS

SEQ ID NO: 8 - protein, 7 aa, Oryctolagus cuniculus
LASTLAS

SEQ ID NO: 9 - protein, 10 aa, Oryctolagus cuniculus
LGGYTGDMYA

SEQ ID NO: 10 - protein, 114 aa, Oryctolagus cuniculus
QSLEESGGRLVTPGTPLTLTCTVSGIDLSTYAMTWVRQAPGKGLEWIGIIYNNGVVVYASWAKGRFTISKTSTTVDLKMTSPTTEDTATYFCCRGYYDGSVNVWGPGTLVTVSL

SEQ ID NO: 11 - protein, 108 aa, Oryctolagus cuniculus
QVLTQTASSVSAVVGGTVTISCQSSQSVYNDNVLSWFQQKPGQPPKLLIYLASTLASGVPSRFSGSGSGTQFTLTISDLECDDAAAYYCLGGYTGDMYAFGGGTEVVV